(19) United States
(12) Patent Application Publication
(10) Pub. No.: US 2024/0013886 A1
(43) Pub. Date: Jan. 11, 2024

(54) METHOD AND SYSTEM OF SIMULATIONS FOR PERSONALIZED BRAIN TREATMENTS

(71) Applicant: Hamed Yousefiroshan, Calgary (CA)

(72) Inventor: Hamed Yousefiroshan, Calgary (CA)

(21) Appl. No.: 18/022,162

(22) PCT Filed: Sep. 9, 2020
(86) PCT No.: PCT/IB2020/058396

§ 371 Date: Feb. 19, 2023

(51) Int. Cl. G16H 20/30

(52) U.S. Cl. CPC G16H 20/30

(57) ABSTRACT

Systems and methods provide a novel approach for decision making and planning of neurovascular treatments and high-fidelity outcome prediction of every potential treatment. The invention, particularly, uses the clinical data of the patients for a personalized treatment planning and simulation in which a personalized anatomical model of the patient is constructed virtually, neurovascular device implantation may be done virtually and by simulation, a computational fluid dynamics (CFD) simulation is done, and finally by using some post-processing parameters, indices and principles a prediction is made regarding the outcome of each potential treatment. The system comprises one or more processors to receive patient-specific data regarding a geometry of an anatomical structure of the patient and to simulate deployments of different neurovascular devices and their corresponding hemodynamics in anatomical structure models and to generate a report for each potential deployment.

### TECHNICAL FIELD

Embodiments for the present invention are methods and systems for modeling of brain flow dynamics, modeling of the related medical devices, and outcome prediction of applying each treatment including but not limited to applying medical devices. More specifically, the present invention and its embodiments include methods and systems for personalized modeling of hemodynamics for brain aneurysms, modeling of the related aimed medical devices for cerebral aneurysms treatment, and outcome prediction of applying each treatment including but not limited to medical devices via hemodynamics simulation and/or medical devices simulation and their post-processing interpretations.

### BACKGROUND ART

Aneurysmal subarachnoid hemorrhage (aSAH), responsible for about 4% of all strokes, is a life-threatening disease that occurs frequently in the most productive years of adults (40-60 age), due to aneurysm rupture and can be diagnosed via symptoms and screening, and prevented by minimally invasive treatments. Aneurysms are bulging portions over the surface of arteries that occur generally due to high blood pressure at weak points of artery walls and may rupture causing aSAH.

Implantation of intra-saccular biocompatible coils within the aneurysmal volume has been at the core of minimally invasive therapies via angiography catheterization, widely accepted after Gulliami FDA approval. This will trigger intra-saccular thrombosis and may lead to the healing of the aneurysm.

Stent-assisted coiling is the implantation of a high-porosity stent within the parent arteries suffering from wide-neck aneurysms, essentially to prevent the protrusion of coil into the parent artery and embolism.

A more recent update for a minimally invasive treatment is the low-porosity stent, so-called flow diverting (FD) stents. FDs have become another preferred minimally invasive option since the 2011 FDA approval of Pipeline. These stents are placed in an extra-saccular fashion, within the parent artery, and were named flow diverters since they attempt to divert blood from aneurysmal volume to the parent artery, in hopes of platelet stimulation, gradual mature thrombus formation in the sac followed by accumulation of smooth muscle cells over the neck region, and a final neointima layer formation; thus eliminating the aneurysm from the circulation.

### Technical Problem

Unfortunately, there is not a unifying reliable standard-of-care for recognition of a possible aSAH since both small (<10 mm) and large aneurysms are prone to rupture regardless of their sizes. Although FDs and coils have been facilitating clinical interventions, eliminating the need for high-volume centers for high-risk open surgeries, their widespread utilization by clinicians must be meticulously verified concerning long-term clinical results. The fundamental fact that the majority of intracranial aneurysms do not rupture throughout the life of the diagnosed individuals has to be considered when an intervention is planned so to avoid the potentially detrimental consequences of these minimally invasive treatments including but not limited to an average of 7% intra-operative mortality rate, post-operative sudden/late rupture, recanalization, in-stent stenosis, migration of implanted devices and prolonged-in-session or follow-up repeated carcinogenic X-ray exposures.

Numerous researchers [see NPL1-5] and inventors [see PLT1-3] both in engineering and in clinical practice have been attempting to predict the outcomes of endovascular treatments for brain aneurysms over several years. Furthermore, Computational Fluid Dynamics (CFD) can be a reliable tool for analyzing blood flow in brain aneurysms and understanding whether blood flow pattern after FD implantation is favorable or not. However, there are various methods for device deployment and CFD simulations, and a variety of parameters to interpret the results of the flow dynamics simulations. In terms of accuracy and reliability, there is currently no established gold standard for outcome prediction of intra/extra-saccular devices implantations for brain aneurysms treatment.

### Technical Solution, Advantage, and Industry Applicability

The systems and methods of the present invention include a novel and precise framework of “personalized fast accurate virtual medical device implantation, CFD simulation, and flow dynamics post-processing for patient treatment decision-making” to establish a gold standard for the optimum treatment planning of intracranial aneurysms; It is available for all neurovascular devices including but not limited to stents and coils. Herein, we show the proof of concept of technology for putting preventive actions into effect to avoid aSAH and/or a badly planned potentially detrimental treatment; reliable and optimal decision-making is determined via personalized pre-session computational simulation and utilized to for preventive management of aSAH cases. The case-specific three-dimensional (3D) models of aneurysms derived from each patient's updated clinical database a computerized customized simulation. Pre-session calculations eliminate lengthy in-session trial and error procedures such as checking for blood flow stasis after multiple FD implantations while the patient is being exposed to radiation, as well as the elimination of irreversible procedures like implantation of an FD before coiling or detrimental implantations of FDs or coils. This invention introduces a fundamentally novel set of mechanistic principles and indices to obtain healing of cerebral aneurysms after intra-(luminal/saccular) procedures. The present methods and systems are different from existing approaches in many aspects; it predicts whether or not complete healing of aneurysms happens on a daily basis with hundred percent accuracy in our series (see Detailed Description), the possibility of sudden/late ruptures, collateral occlusions, in-stent stenosis and provides recommendations for antiplatelet drug dosage-reduction/cessation. The method and systems disclosed herein can easily be applied in angiography sites in medical imaging apparatus or a computer system or out of angiography sites by using a computer system.

### SUMMARY OF INVENTION

This invention provides simulation and treatment outcome prediction for any type of treatment of brain aneurysms. The treatments may include utilizing any device in any format. Examples of the devices presented here will not limit the scope of this invention and are just for clarification. Various new devices may be designed and manufactured in prototype, animal, or final human clinical formats based on the systems and methods provided by this invention. The section entitled “Detailed Description,” more reveals the unique benefits and capabilities of this invention over similar ones in terms of accuracy, feasibility, and speed.

According to an aspect of this invention, a system for simulation of final deformed deployed shape and configuration of neurovascular devices and their corresponding hemodynamics in anatomical structure models and post-processing is provided. The system comprising:
a database configured to store neurovascular device characteristics and,
the processor(s);
The processor(s) is configured to virtually construct a part or whole of the anatomical structure models of a patient, virtually place a plurality of the neurovascular devices in the anatomical structure models, simulate the blood flow dynamics after the virtual placement of the plurality of the neurovascular devices in the anatomical structure models, and calculate the post-processing parameters, indices, and principles for interpretation and reporting the outcome of the treatment.

According to an aspect of this invention, a method for simulation of final deformed deployed shape and configuration of neurovascular devices is provided.

The method comprising:
a database configured to store neurovascular device characteristics and,
the processor(s);

The processor(s) is configured to virtually construct a part or whole of the anatomical structure models of a patient, virtually place a plurality of the neurovascular devices in the anatomical structure models and simulate the blood flow dynamics after the virtual placement of the plurality of the neurovascular devices in the anatomical structure models. The anatomical structure model comprising blood vessel(s) and at least one velocity magnitude of blood within the blood vessel(s). In this embodiment, the method may consist of receiving a selection of neurovascular device characteristics from a collection stored in a database, virtual placement, by the processor(s), of the selected devices in the anatomical structure models, and simulation of hemodynamics after device placements.

Another aspect of this invention is providing a novel computational and a novel computational post-processing method for simulation and prediction of the possible outcomes regarding various neurovascular device implantations. The invention virtually places the devices in the personalized anatomical structure(s), simulates the precise neurovascular device deformations and final configurations, and simulates, by computational fluid dynamics (CFD), the corresponding hemodynamic outcomes, and provides high-fidelity treatment outcome predictions by the post-processing feature.

In another embodiment of this invention, a system of cloud-based data processing may be used. This embodiment may utilize a computer cluster for receiving patient data by utilizing a User Interface (UI). A three-dimensional model of the anatomical structure is constructed out of the patient clinical data. A plurality of device characteristics may be received by the computer cluster from a server. The final deformed post-implantation configuration of devices may be constructed using the Babol Method as described in the section entitled “Detailed Description”. A user may select the device characteristics from the database already stored in the server. The tasks of the computer cluster may comprise:

To virtually place, the final deformed shape of neurovascular devices in the anatomical structure models, simulate the anatomical structure model mesh, simulate hemodynamic outcomes using computational fluid dynamics, and performing the corresponding post-processing results for treatment outcome predictions.

Another embodiment of this invention provides a computerized method that may utilize a computer cluster for receiving patient clinical data. A three-dimensional model of the anatomical structure may be constructed out of the patient data. A plurality of device characteristics as a database may be stored by the method. The final deformed post-implantation configuration of device models may be constructed using Babol Method as described in the section entitled “Detailed Description”. A user may select the device characteristics from the database already stored in the server. The tasks of the computer cluster may comprise:

To virtually place, the final deformed post-implantation configuration of neurovascular devices in the anatomical structure models, simulate the anatomical structure model mesh, simulate hemodynamic outcomes using computational fluid dynamics, and performing the corresponding post-processing results for treatment outcome predictions.

In all embodiments of this invention:

The anatomical structure model may comprise a computational model,
initial or the deformed shape of the device(s) after implantation may include volume mesh and a computer-aided design (CAD) geometry,
may comprise blood volume mesh(es) from the meshing of the selected device characteristics and the anatomical structure model,
the computer cluster may virtually place a plurality of devices in the anatomical structure model.

### DETAILED DESCRIPTION

Herein, many special details of this invention are presented to provide a complete understanding of the different aspects of the invention. More general and known arrangements, relations, and devices are presented or explained just for clarification purposes; skilled persons in the related arts may find no need for these details to apply the invention. The representation of the operation is enough to enable persons to put the different forms of the invention into effect, especially for software implementation. Furthermore, there are various and alternative devices, arrangements of elements, and technologies that the presently disclosed invention can be used for. Examples presented here, are just for clarifications and the full scope of the invention will not be bounded by them.

The system may be performed by a computer, computer cluster, processor, or server according to the descriptions as follows. The system implements a method of treatment outcome prediction and treatment planning for a patient in a personalized fashion, particularly in terms of the effects of the various device deployments on the healing of brain aneurysms. The method can be applied by one or multiple software modules or a combination of them run by one processor or more processors. In some embodiments, the system steps are done manually; they can be repeated by the user choosing different neurovascular devices. Also, the system can automatically do a simulation of the final deformed deployed shape and configuration of different neurovascular devices and/or different sizes of a neurovascular device. For example, several different types and sizes of neurovascular self-expanding stents may be virtually placed in a parent artery suffering from aneurysm(s) that may lead to different outcomes; the user, e.g. a physician, may enter some indications and the system may automatically simulate deployment and verify the suitability of a specific device as the desired treatment suggested by the user. The system may test a plurality of treatment options, including applying various devices and sizes and may report or suggest an optimum option as an output. In other embodiments, the system can verify various devices or their sizes without receiving any input from the user. In all possible embodiments, the system may give a report as an output at the end of the simulations. FIG. 1 is a schematic representation of the whole process.

The realistic three-dimensional model of aneurysm and parent artery

Three-dimensional, could be but not limited to stereolithography (STL), models of aneurysms, and parent arteries are extracted out of patient's clinical data using 3D-Slicer software (www.slicer.org).

Accurate Virtual Implantation of Stents

A two-dimensional view of the model is selected. To obtain an accurate final microscopic, deformed configuration of the stent after implantation, an approach, named “Babol Method”, is introduced as follows:
I. Any FSI (Fluid-Structure-Interaction) between blood and stent and frictional forces between stent wires is negligible.
II. It is enough to consider only the region under the neck of the aneurysm, unless an adjacent perforator is present, or when needed regarding “Eshrat Principles of Occlusion, (EPO)” as will be discussed later.
III. Considering a single wire of a stent with the weaving angle of 75° (or the weaving angle defined by the manufacturer), deployed in an artery of diameter “d”, the free-state (fully unsheathed) diameter of the stent is D_{fS}. The α angle (FIG. 2c) of the deformed wire after implantation with respect to the long axis of the stent is

$${sin}^{- 1} ( \frac{d}{l} ) ,$$

where

$$l = \frac{{D}_{f s}}{sin 75}$$

IV. Herein, for the first time, we formulate the transition and compaction lengths of stents under the neck of aneurysms [NPL6]. By using a two-dimensional bounding box (FIG. 3a), the stent is bent and adapted according to the parent artery wall geometry. This is done by introducing a new function for all neurovascular self-expanding stents called “Isa Function”. Isa is defined as Isa=f(N, t, PF, Lₜ):

PF is the Perimeter Fullness; the quantity of the perimeter of an imaginary circle for the free-state FD that is covered by any lateral cross-section of the stent, e.g. a Pipeline Embolization Device (PED) with 48 wires of 26 μm makes a 1.248 mm length in any lateral cross-section, Therefore, for a 5.0 labeled PED (D_{fs}=5.25 mm), the corresponding PF value is 0.076,
t is the thickness of each wire of stent; for a PED, t is 26 μm, but for a Derivo Embolization Device (DED) 35 μm,
N is the number of stent wires.

The roles of N, t, and PF are schematized in FIG. 2b,

The formula for transition lengths:

The difference in volume due to the difference in length between D_{fs} and d should be compensated by a change in volume between the distal and proximal tips of the aneurysm; for certain values, the quantity of the perimeter of the free-state stent would be equal to the quantity of the volume of a cylinder having the diameters of the parent artery at distal or proximal locations. Hence:

$$\frac{π}{4} {d}^{2} {L}_{t} = π {D}_{f s}$$

$${L}_{t} = \frac{4 {D}_{f s}}{{d}^{2}}$$

For 2<d<3 mm, Makoyeva et al [NPL6] suggested, experimentally, a linear relationship of Lₜ for a 3.5 labeled flow diverter (FD). The equation for Lₜ in this invention, at d=3 mm, and D_{fs}=3.75 mm, (a 3.5 labeled FD), gives an Lₜ of 1.67 mm, which is exactly the same as the experimental value (FIG. 4). If d<3 mm we use a linear relationship, and if d>3 mm the Lₜ Equation. The line of 2<d<3 mm is horizontally offset to hit profiles of Lₜ the equation for different PEDs at Lₜ=1.67 mm. Accepting the experimental line as a reference, all other lines should rotate at the hit points with respect to it counterclockwise (if they have greater PF values), or clockwise (for smaller PF values). As an example, for a nominal diameter of 5.0 mm, i.e. the free-state diameter of 5.25 mm, the difference in slopes of the lines according to the corresponding PF values would be (0.106-0.076)=0.03, which gives us:

Δθ=θ_{D}_{ₙₒₘ₌₅}−θ_{D}_{ₙₒₘ}=3.5=tan⁻¹(0.03)=1.72°(Clockwise)

For Dnom<d<D_{fs} we use a linear relationship (Lₜ=0 for d=D_{fs}). (FIG. 4).
IV. The stent reaches its free-state diameter unless there is not enough room of Lₜ, or the parent artery is not straight, i.e. curved. If there is not enough room for Lₜ, the same percentage reduction proportionate to the reduction of Lₜ will be considered both for Lₜ and D_{final}, the maximum final diameter of the stent.
V. The center of rotation of the parent artery is calculated. Perpendicular bisectors with the quantities of different Lₜ for inlet and outlet sections, and a section with D_{final}, are drawn. The section's center is attached to the end tip of the Lₜ line oriented towards the center of rotation of the parent artery. The gaps between the tip of the drawn section and the wall of the artery (inward or outward, lines in FIG. 2a) are added to D_{final}. If the gap is outward, it is a negative quantity; if inward, it will be positive. The resulting D_{final} will be constant throughout the compaction zone—between the two transition zones. The diameter of the stent at the inlet/outlet is calculated based on the gaps, i.e. for an inward gap, the diameter will be suppressed accordingly, but if outward, the diameter of the stent at the inlet/outlet will not change. Sections (diameters) for transition zones are calculated linearly from the proximal/distal sections to the either beginnings of the compaction zone. More cross-sections lead to more preciseness.
VI. To obtain 2D sketches of strands patterns, lines perpendicular to the bottom tip of sections are drawn (FIG. 2c). Two-dimensional clockwise and counterclockwise lines are sketched from distal and proximal locations, separately, until they meet each other at the beginning of the compaction zone. Initially, the angles of these lines are based on a and p. To calculate a, the quantity of “d” would be the diameter of each section obtained from step V. In straight vessels both lines have an identical a degree with respect to the perpendicular line at the bottom tip of the section, making a 2a angle, while for curved arteries we assume the upper lines maintain a, and the lower lines take the angle β which is “a plus the angle between the two neighboring sections”. This is to put rotation into effect. At any section, exactly, 11 hits must be seen by clockwise/counterclockwise lines, because in a non-confined stent this is the case. Forever, first of all, the lower lines are sketched and offset to hit the next neighboring section at 11 points; for the first section, the upper lines are attached to the hit points, starting with the first upper line reaching the nearest hit point on its direction; i.e. a may change but p remains constant between 1st and 2nd sections. To continue with the 2nd section, both a and p are subject to change. The first lower line generated by offset is attached to the nearest hit point on section 2 on its direction; the first upper line that is at the bottom tip of the 2nd section will be ignored, and a parallel line will be replaced at the first hit point of section 2, which targets the nearest hit point on the 3rd section on its direction.
VII. If multiple stents are deployed, three superposition patterns of fully-, partial- and half-overlapped are used, and each pattern will be considered and analyzed for hemodynamics analysis, separately.
VIII. If non-standard stent deployments like axial compressions (Push-Pull technic) of the stents are applied, unique and new transition lengths will be considered for the compaction zone, beginning from the end of each transition length. The compaction zone, in this way, will be divided into four zones, i.e. the two new transition zones in the compaction zone (TZC) and two super-compacted zones (SCZ). The α angle at the beginning of the SCZ and the TZC would be 75 (or the weaving angle defined by the manufacturer), and an average of “75 (or the weaving angle defined by the manufacturer) plus the original a at the end section of the main transition zone”, respectively.
IX. A three-dimensional “bed” of the final deformed FD can be obtained from the previous steps onto which all 2D sketches are projected. Three-dimensional strands will be available by giving appropriate thicknesses to the projected lines on the 3D bed.

Validation of Babol Method (Virtual Implantation of Stents)

To validate the simulation for the final deformed shape of stents after implantations, numerous cases were rigorously tested; among them, two cases are shown herein. Metal coverage is shown for a real [NPL7], and for a straight glass tube case (FIG. 3 and FIG. 5). The same formula adopted by each study were used separately, with the wire thickness of 26 μm, and 30 μm based on the studies, respectively. Pore densities for the real case were calculated in the same way done by the authors. Another important parameter is the quantities of the diameters of FD between the proximal and the distal tips of the aneurysm under the neck (FIG. 6). These quantities were calculated for Babol Method for the real case. Measurements were made with Shapr3D (www.shapr3d.com), and ImageJ (National Institutes of Health, Bethesda, Maryland) for further checking. As could be seen, the virtual implantation method of stents presented in this invention (Babol Method), though superfast and easy, is in very good agreement with the realistic microscopic, deformed shape of the stent.

Hemodynamics Analysis via CFD Simulation

As shown in FIG. 2, inlet and outlet(s) are considered for the parent artery of the aneurysm(s). Navier-Stokes and continuity equations for laminar steady-state flow are solved by using the first-order finite element solver SimVascular [NPL8] based on the zero pressure for the outlets. No smoothing performed on STL models to maintain the originality of the anatomies as much as possible. No extended entrance lengths were added to STL 3D models. Average mesh independency values are 2.7 and 11 million tetrahedral elements for non-stented and stented cases, respectively. Blood is considered Newtonian, incompressible, and arteries solid with the no-slip boundary condition, the density of blood and dynamic viscosity, 1060 (kg/m3), and 0.003 (Pa·s), respectively. Regardless of the location of aneurysms, for all inlets, a 60 cm/s velocity magnitude is considered. Hemodynamics is assessed based on the LAKE-MAKE Theory, EPO, and Yousefiroshan index as follows to prognosticate the stent or coil implantation outcomes. Note that for coiled cases no simulation is run, and just pre-coiled hemodynamics is enough to judge about the outcome of coiling.

Post-processing Parameters, Indices, and Principles

LAKE-MAKE Theory

Two new parameters are introduced for prediction of the status and time of a possible occlusion of aneurysms after stent or coil placement as follows:

$$M A K E = \frac{{\sum }_{i = 1}^{n} ( {u}_{i}^{2} + {v}_{i}^{2} + {w}_{i}^{2} )}{n}$$

MAKE as an approximation for Magnitude of Averaged Kinetic Energy, is a representation of the intensity of dynamic flow in aneurysmal volume. The volume is divided into “n” nodes, and each node possesses velocities of u, v, w in the x, y, z directions in space.

$$L A K E = \frac{{\sum }_{i = 1}^{n} [ {x}_{i} , {y}_{i} , {z}_{i} ] ( {u}_{i}^{2} + {v}_{i}^{2} + {w}_{i}^{2} )}{M A K E}$$

LAKE as an approximation for Location of Averaged Kinetic Energy is a representative point for the whole blood flow in the aneurysmal volume where kinetic energy is concentrated. Considering the height of an aneurysm, LAKE possesses values between zero to unity, i.e. if LAKE is a point located in the middle of the distance from the ostium area center to the dome of the aneurysm, its quantity will be 0.5.

Eshrat Principles of Occlusion (EPO)
I. Kinetic energy reduction is necessary but not enough for aneurysm occlusion after stent implantation.
II. Aneurysm must not be a feeder. After stent implantation, no physical points, including perforators and branches or other aneurysms, out of the aneurysmal volume are allowed to receive blood directly from the aneurysmal volume, immediately after exiting of blood from the aneurysmal volume; i.e. a whole (e.g. a single vortex is the only flow structure in the aneurysmal volume) or a portion of the blood flow (e.g. one out of two or more separate vortices in the aneurysmal volume) must not circulate in the aneurysmal volume and finally exit from the aneurysmal volume and immediately and directly feed any point external to the aneurysmal volume. Otherwise, it will be interpreted as a failure regarding aneurysm occlusion (FIG. 7).
III. In the absence of a sudden rupture after stent placement, aneurysm occlusion occurs if and only if EPO I and EPO II principles are met. The corresponding occlusion time would be as follows:

Yousefiroshan Index: Sixty percent reduction in MAKE along with a LAKE of 0.5 is associated with 180 days occlusion. Considering this status as a base, any decrease/increase of MAKE or LAKE proportionally makes changes to the time of occlusion. For example, a 75% reduction of MAKE with a LAKE of 0.5 will result in 180(1−0.15)=153 days of complete occlusion; if LAKE is also 0.25 instead of 0.5, then 180(1−0.4)=108 days is expected for the aneurysm to be closed completely.

Note: Regardless of any LAKE quantity, a less than 20% MAKE reduction after stent implantation is interpreted as an unfavorable condition for a one-year follow-up, even if LAKE is less than 0.1.

Validation of the Prediction Feature

Twelve stented and nine coiled real aneurysm cases from multiple centers were analyzed against the prediction feature of this invention based on CFD simulations, EPO, LAKE-MAKE, and Yousefiroshan index. All cases were predicted blindly, i.e. the pre-implanted clinical data of the patients along with the characteristics of the utilized FDs (brand, diameter, length) were delivered to the inventor by the physicians without firstly reporting the outcomes of the treatments to the inventor. All stented cases were predicted with the excellent hundred percent accuracy based on the months of occlusions or no-occlusion in a one-year follow-up. Also, all coiled cases were predicted accurately if coiling was inefficient for the complete occlusion of the aneurysms for a one-year follow-up.

Any device set forth herein may be used in any suitable medical procedure, and advanced through any suitable body lumen and body cavity and may be used for any suitable part of the body. Any feature or aspect outlined in any embodiment may be used with any other embodiment set forth herein.

Any modifications or variations may be made by the skilled people in the related art without departing from the scope of this disclosed invention which will be more apparent by what is claimed for this invention.

Wherever is in this text or claims, unless otherwise explicitly stated, the word “comprise”, and variations such as “comprises” and “comprising”, is understood to imply the inclusion of a stated integer or step or group of integers or steps but not the exclusion of any other integer or step or group of integers or steps.

Statement of any references or publications or any other matter or any information derived from them does not mean an acknowledgment or admission or any form of suggestion that references or publications or any other matter or information derived from them forms part of the common general knowledge in the field of endeavor to which this specification relates.

Wherever is in this text or claims, unless otherwise explicitly stated, the word “processor” is to be given its ordinary and customary meaning to a person of ordinary skill in the art. A processor may be a computer system, tablet, smartphone, smartwatch, Pad, Phone, laptop, state machine, processor, or anything that does the task of arithmetic or logic operations using logic circuitry that responds to and processes the basic instructions that drive a computer. The processor may refer to ROM and/or RAM in some embodiments.

Headings will not limit the scope of the invention and are just presented to help readers for clarifications and better understanding.

### BRIEF DESCRIPTION OF DRAWINGS

The drawings and figures are just for clarification purposes and will not limit the scope of the invention.

FIG. 1 is an overview of the whole process and steps of the invention.

FIG. 2a shows any typical aneurysm with the dimensions depicted which is almost identical to the real aneurysm of [NPL7]; FIG. 2b is a representation of various neurovascular stents in their lateral cross-sections; lateral cross-sections of 5.0 & 2.0 mm PED, and 4.5 mm LVIS together with each other in a single plane to illustrate N, PF, and t parameters in Isa function; FIG. 2c shows the definition of various angles used in the invention.

FIG. 3a shows the final deformed configuration of a 3.0 PED deployed in the aneurysm of the FIG. 2a; FIG. 3b shows the comparison of metal coverage and pore density of the deployed stent for the proximal transition (PT), the compaction (or the middle zone, M), and the distal transition (DT) zones, between three approaches of the Babol Method (this invention), experimental and HiFiVS (the finite element method [NPL7]).

FIG. 4 shows the relationship between the length of transition for PEDs of different nominal diameters versus the different diameters of the parent artery. The LVIS (1) line and the LVIS (2) line represent the LVIS experiment's transition length [NPL6] and Babol Method's transition length (this invention), respectively.

FIG. 5a the upper image shows a 4.25×20 mm PED placed in gradually increasing diameter straight tubes (neuroangio.org used with permission), and the lower image shows the same PED simulated with Babol Method (the present invention); FIG. 5b shows a Head-to-Head comparison of Metal coverage between the experiment and Babol Method.

FIG. 6 is a Head-to-Head comparison of diameters for 14 sections under the neck of the aneurysm of FIG. 2a for a 3.0 mm PED, between the experiment, HiFiVS (Finite Element Method) [NPL7], and Babol Method (this invention).

FIG. 7 shows the definition of the feeder (top) and non-feeder (bottom) aneurysms. Blood circulates in the feeder aneurysm, and immediately after exiting the aneurysm, is divided into two ways, in one way directly flows toward an adjacent branch which contrasts with the blood circulation in the non-feeder aneurysm and exiting from it as shown.

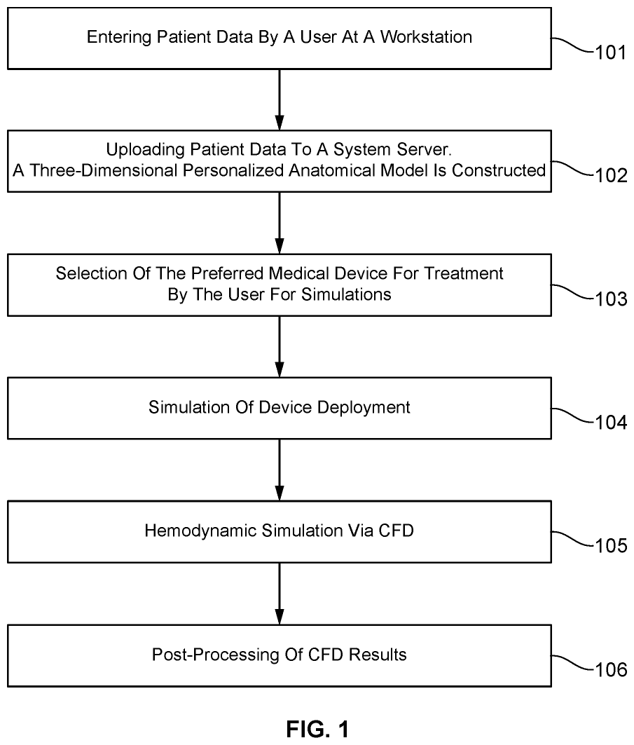

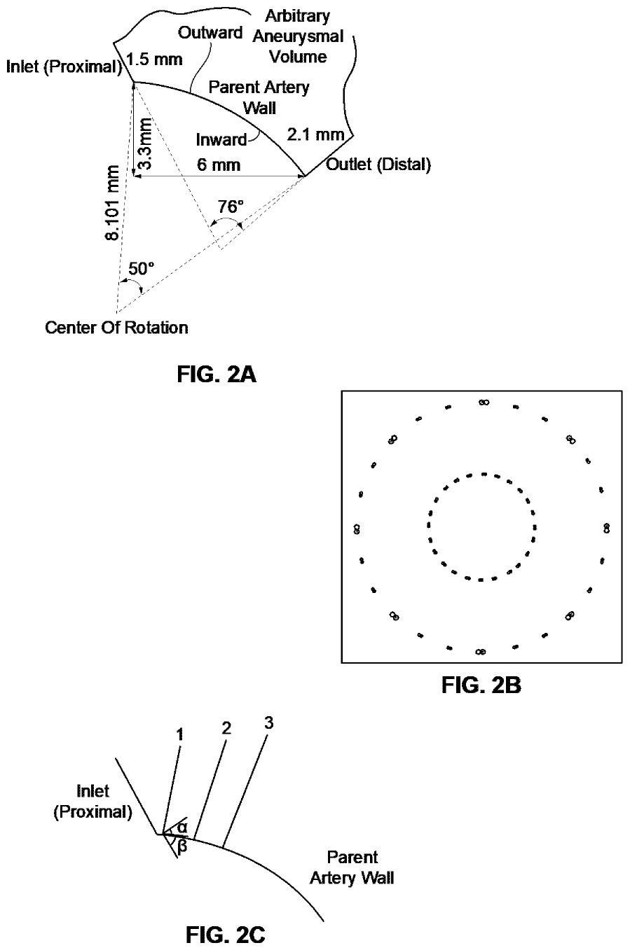

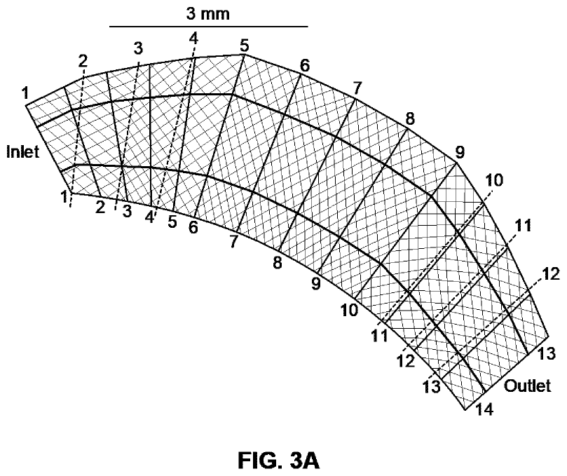

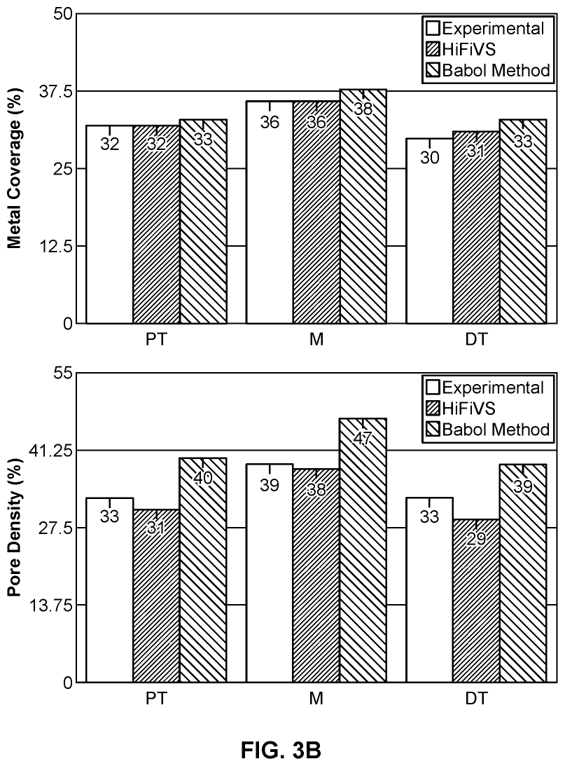

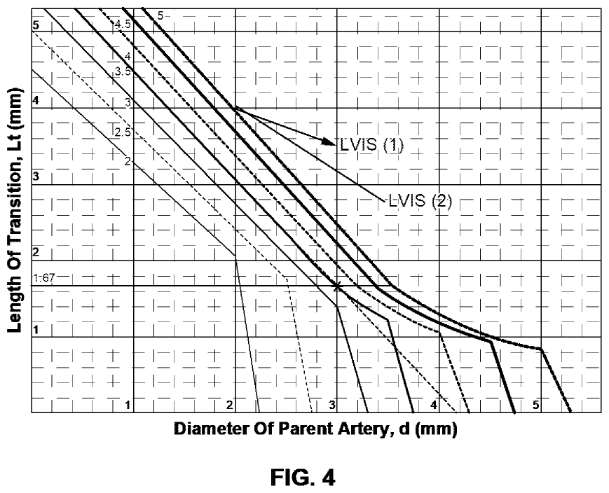

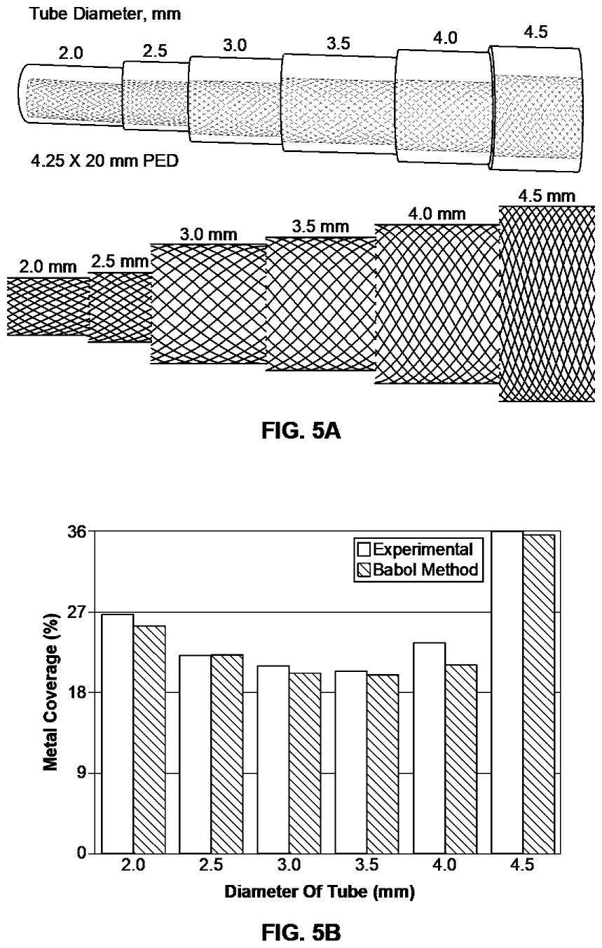

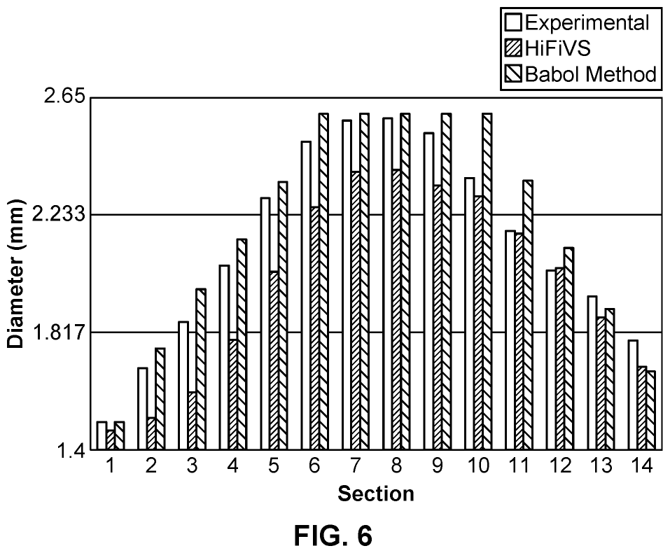

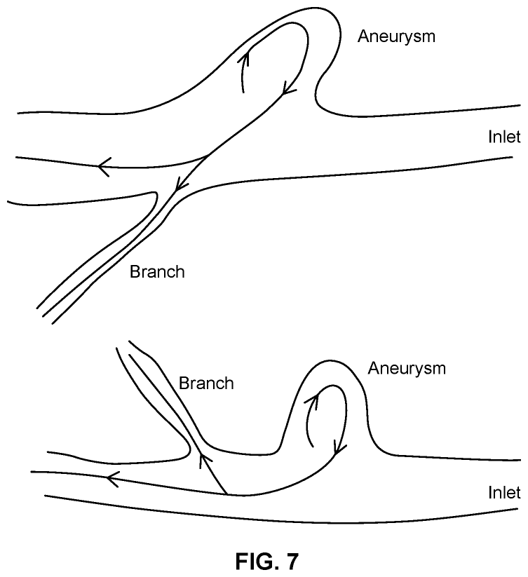

1. A system for simulation of final deformed deployed shape and configuration of neurovascular devices and their corresponding hemodynamics in anatomical structure models, the system comprising:
a database configured to store neurovascular device characteristics of different neurovascular stents, comprising the diameter of the device, length, and the thickness and number of braided strands,
a user interface configured to receive clinical data of a patient, wherein the user interface is configured to allow a user to select a plurality of the neurovascular device characteristics from the database; and
one or more processors configured to:
virtually construct an anatomical structure model of the patient,
virtually construct the final post-implantation deformed shape of the neurovascular device model by:
firstly, making a two-dimensional bounding box comprising the boundaries of the anatomical structure and the boundaries of the final post-implantation deformed shape of the stent obtained by:
calculating at least two transition zones and at least one compaction zone between the distal and proximal tips of the aneurysm(s) under the neck, and,
calculating the center of rotation of the anatomical structure, and,
calculating the maximum final post-implantation diameter of the stent under the neck, and,
calculating the diameters of the deformed stent at the distal and proximal tips of the aneurysm(s),
secondly, by modeling the plurality of braided strands via several two-dimensional clockwise and counterclockwise lines within the bounding box,
simulate the placement of the plurality of the neurovascular device models in the anatomical structure model via:
modeling a three-dimensional bed regarding the dimeters of the deformed stent at the distal, proximal, and compaction zones and,
projection of the two-dimensional lines onto the three-dimensional bed to obtain three-dimensional lines and,
assigning the corresponding thickness of strands to the three-dimensional lines in the bounding box,
generating at least one stent volume mesh and at least one blood volume mesh,
simulation of hemodynamics after simulating the virtual placement of the plurality of the neurovascular device models in the anatomical structure model,
calculating the post-processing parameters, indices, and principles after the hemodynamics simulation,
generate a report comprising one or more of hemodynamics post-processing data regarding the neurovascular device model performance data; and
select a device for use in neurovascular device placement procedure based at least in part on one or more of the hemodynamic post-processing data and the neurovascular device model performance data.

2. (canceled)

3. (canceled)

4. (canceled)

5. The system of claim 1, wherein the anatomical structure model comprises one or more blood vessels or arteries and one or more aneurysms, and wherein the anatomical structure model comprises at least one velocity magnitude within one or more of the blood vessels or arteries.

6. (canceled)

7. (canceled)

8. The system of claim 1, wherein the neurovascular device models comprise one or both of a volume mesh and a CAD geometry.

9. The system of claim 1, wherein the stent is any neurovascular self-expanding stent.

10. The system of claim 1, wherein Isa Function along with at least one correction coefficient is used to determine the lengths of transition zones.

11. The system of claim 1, wherein the formula for the length of the transition is used to determine the lengths of transition zones.

12. The system of claim 1, wherein gap quantities are defined with respect to the center of rotation of the anatomical structure to determine the final maximum diameter of the deformed stent under the neck of the aneurysm(s), and to determine the diameter of the stent at the distal and proximal tips of the aneurysm(s).

13. The system of claim 1, wherein the diameters of the stent in each of the transition zones are assigned by a trendline.

14. The system of claim 1, wherein the angles of the clockwise and counterclockwise lines are determined in the bounding box.

15. The system of claim 1, wherein the post-processing parameters, indices, and principles are used to predict the outcome of any treatment decision regarding utilizing the neurovascular devices.

16. A method for simulation of final deformed deployed shape and configuration of neurovascular devices and their corresponding hemodynamics in anatomical structure models, the method comprising:
storing a computer-readable database comprising different neurovascular stents, comprising diameter of the device, length, and the thickness and number of braided strands; receiving clinical data of a patient,
selecting a plurality of the neurovascular device characteristics from the database, and
by using one or more processors:
virtually construct an anatomical structure model of the patient,
virtually construct the final post-implantation deformed shape of the neurovascular device model by:
firstly, making a two-dimensional bounding box comprising the boundaries of the anatomical structure and the boundaries of the final post-implantation deformed shape of the stent obtained by:
calculating at least two transition zones and at least one compaction zone between the distal and proximal tips of the aneurysm(s) under the neck, and,
calculating the center of rotation of the anatomical structure, and,
calculating the maximum final post-implantation diameter of the stent under the neck, and,
calculating the diameters of the deformed stent at the distal and proximal tips of the aneurysm(s),
secondly, by modeling the plurality of braided strands via several two-dimensional clockwise and counterclockwise lines within the bounding box,
simulate the placement of the plurality of the neurovascular device models in the anatomical structure model via:
modeling a three-dimensional bed regarding the dimeters of the deformed stent at the distal, proximal, and compaction zones and,
projection of the two-dimensional lines onto the three-dimensional bed to obtain three-dimensional lines and,
assigning the corresponding thickness of strands to the three-dimensional lines in the bounding box,
generating at least one stent volume mesh and at least one blood volume mesh,
simulation of hemodynamics after simulating the virtual placement of the plurality of the neurovascular device models in the anatomical structure model,
calculating the post-processing parameters, indices, and principles after the hemodynamics simulation,
generate a report comprising one or more of hemodynamics post-processing data regarding the neurovascular device model performance data; and
select a device for use in neurovascular device placement procedure based at least in part on one or more of the hemodynamic post-processing data and the neurovascular device model performance data.

17. (canceled)

18. (canceled)

19. (canceled)

20. The method of claim 16, wherein the anatomical structure model comprises one or more blood vessels or arteries and one or more aneurysms, and wherein the anatomical structure model comprises at least one velocity magnitude within one or more of the blood vessels or arteries.

21. (canceled)

22. (canceled)

23. The method of claim 16, wherein the neurovascular device models comprise one or both of a volume mesh and a CAD geometry.

24. The method of claim 16, wherein the stent is any neurovascular self-expanding stent.

25. The method of claim 16, wherein Isa Function along with at least one correction coefficient is used to determine the lengths of transition zones.

26. The method of claim 16, wherein the formula for the length of the transition is used to determine the lengths of transition zones.

27. The method of claim 16, wherein gap quantities are defined with respect to the center of rotation of the anatomical structure to determine the final maximum diameter of the deformed stent under the neck of the aneurysm(s), and to determine the diameter of the stent at the distal and proximal tips of the aneurysm(s).

28. The method of claim 16, wherein the diameters of the stent in each of the transition zones are assigned by a trendline.

29. The method of claim 16, wherein the angles of the clockwise and counterclockwise lines are determined in the bounding box.

30. The method of claim 16, wherein the post-processing parameters, indices, and principles are used to predict the outcome of any treatment decision regarding utilizing the neurovascular devices.
